(19) United States
(12) Patent Application Publication
(10) Pub. No.: US 2016/0354193 A1
(43) Pub. Date: Dec. 8, 2016

(54) PROSTATIC URETHRAL STENT

(71) Applicant: Bruce Gordon McNAB, SR., Maplecrest, NY (US)

(72) Inventor: Bruce Gordon McNAB, SR., Maplecrest, NY (US)

(21) Appl. No.: 14/994,919

(22) Filed: Jan. 13, 2016

Related U.S. Application Data
(60) Provisional application No. 62/171,400, filed on Jun. 5, 2015.

(51) Int. Cl. A61F 2/04; A61F 2/94

(52) U.S. Cl. CPC A61F 2/04; A61F 2/94; A61F 2002/047; A61F 2310/00023

(57) ABSTRACT

A prostatic urethral stent for the nonsurgical regulation of prostatic urethral flow is provided. The prostatic urethral stent has a tubular body terminating in opposing, cone-shaped ends. The prostatic urethral stent is dimensioned and adapted for transurethral insertion into the lumen of the internal urethra sphincter so as to be retained therein by the constrictive forces of the internal urethra sphincter and/or enlarged, surrounding prostate. The cylindrical opening of the tubular body facilitates the urethral flow through the prostatic urethra, wherein the cylindrical opening is maintained by the strength of the tubular body, while the retention of the tubular body is a balance of the constrictive forces of the prostatic urethra and the stiffened shape of the tubular body.

### CROSS-REFERENCE TO RELATED APPLICATION

This application claims the benefit of priority of U.S. provisional application No. 62/171,400, filed 5 Jun. 2015, the contents of which are herein incorporated by reference.

### BACKGROUND OF THE INVENTION

The present invention relates to medical devices and methods thereof and, more particularly, to a prostatic urethral stent for the nonsurgical regulation of prostatic urethral flow.

The prostate is a walnut-sized gland lying inferior to the bladder and surrounding approximately the first inch of the urethra. In older males, it is not uncommon for a progressive enlargement of the prostate to constrict the prostatic urethra, resulting in restricted urine flow there through.

The current treatments for constricted urine flow resulting from an enlarged prostrate have substantial disadvantages One such treatment, Transurethral Resection damages the urethra where it passes through the prostrate, and requires 2-3 days hospital admittance. Other current treatment includes a Thermal application which tends to burn the urethra, as does a Laser Procedure which is also used. Such treatment and procedures risk the possibility of infection and may still require prescription medication, which have their own inherent risks. Moreover, if such treatments fail, because of their invasive nature, or The Prostrate becomes Cancerous requiring it's removal the urethra would have to be reconstructed.

As can be seen, there is a need for a prostatic urethral stent for the nonsurgical regulation of prostatic urethral flow.

### SUMMARY OF THE INVENTION

In one aspect of the present invention, a prostatic urethral stent includes a tubular body extending between opposing ends; a tapered portion near each end, wherein a circumference of each tapered portion decreases as the tapered portion extends toward an associated end; and a cylindrical opening provided through the tubular body, wherein the tubular body is formed from a sufficiently rigid, uric acid resistant, and non flexible material.

In another aspect of the present invention, the prostatic urethral stent includes a tubular body extending between opposing ends for a length that ranges between approximately 3.00 to 3.25 centimeters, wherein a circumferential diameter of the tubular body is approximately 9 millimeters; a tapered portion near each end, wherein a circumference of each tapered portion decreases as the tapered portion extends toward an associated end so that each end has a cone shape; and a cylindrical opening provided through the tubular body, wherein the cylindrical opening has a diameter of approximately 7 millimeters, wherein the tubular body is formed from a material that is resistant to uric acid and compatible with the urethra, and wherein the tubular body is dimensioned and adapted for transurethral insertion into and retention within a lumen of an internal urethra sphincter of a human user.

In yet another aspect of the present invention, a method of non-surgically regulating a urethral flow through a prostatic urethra includes the step of transurethrally inserting the above-mentioned prostatic urethral stent into a lumen of an internal urethra sphincter of a human user.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is a top plan view of an exemplary embodiment of the present invention;

FIG. 3 is a side view of an exemplary embodiment of the present invention;

FIG. 4 is a section view of an exemplary embodiment of the present invention, shown in use (implanted), taken along a medial anatomical plane; and

FIG. 5 is a section view of an exemplary embodiment of the present invention, shown in use (implanted), taken along a posterior anatomical plane.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a prostatic urethral stent for the nonsurgical regulation of prostatic urethral flow. The prostatic urethral stent has a tubular body terminating in opposing, cone-shaped ends. The prostatic urethral stent is dimensioned and adapted for transurethral insertion into the lumen of the internal urethra sphincter so as to be retained therein by the constrictive forces of the internal urethra sphincter and/or enlarged, surrounding prostate. The cylindrical opening of the tubular body facilitates the urethral flow through the prostatic urethra, wherein the cylindrical opening is maintained by the strength of the tubular body, while the retention of the tubular body is a balance of the constrictive forces of the prostatic urethra and the stiffened shape of the tubular body.

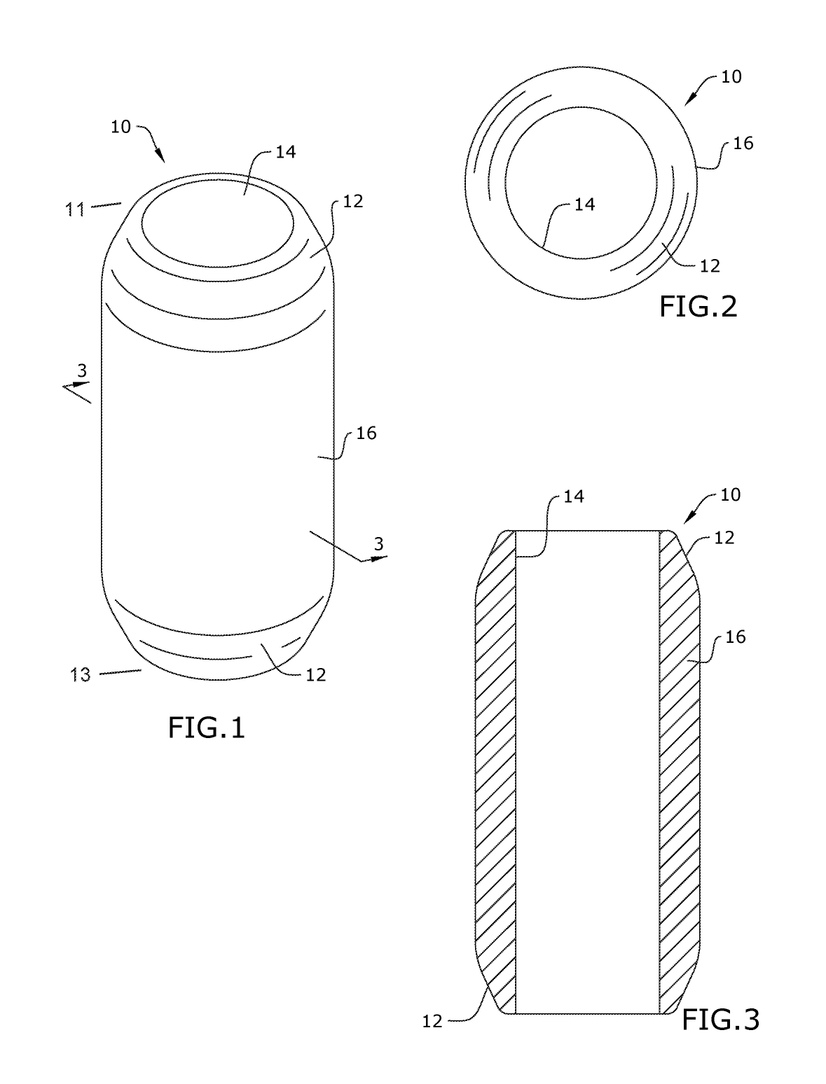

Referring to FIGS. 1 through 3, the present invention may include a prostatic urethral stent 10 for the nonsurgical regulation of prostatic urethral flow 26. The prostatic urethral stent 10 may be a tubular body 16 extending from a first end 11 to an opposing second end 13. The tubular body 16 may have a uniform circumference until a tapered portion 12 near each end 11, 13. The tapered portion 12 may have a decreasing circumference as it extends toward an associated end 11 or 13, thereby giving each end 11, 13 a coned shape. The prostatic urethral stent 10 may provide a uniform cylindrical opening 14 from the first end 11 to the second end 13.

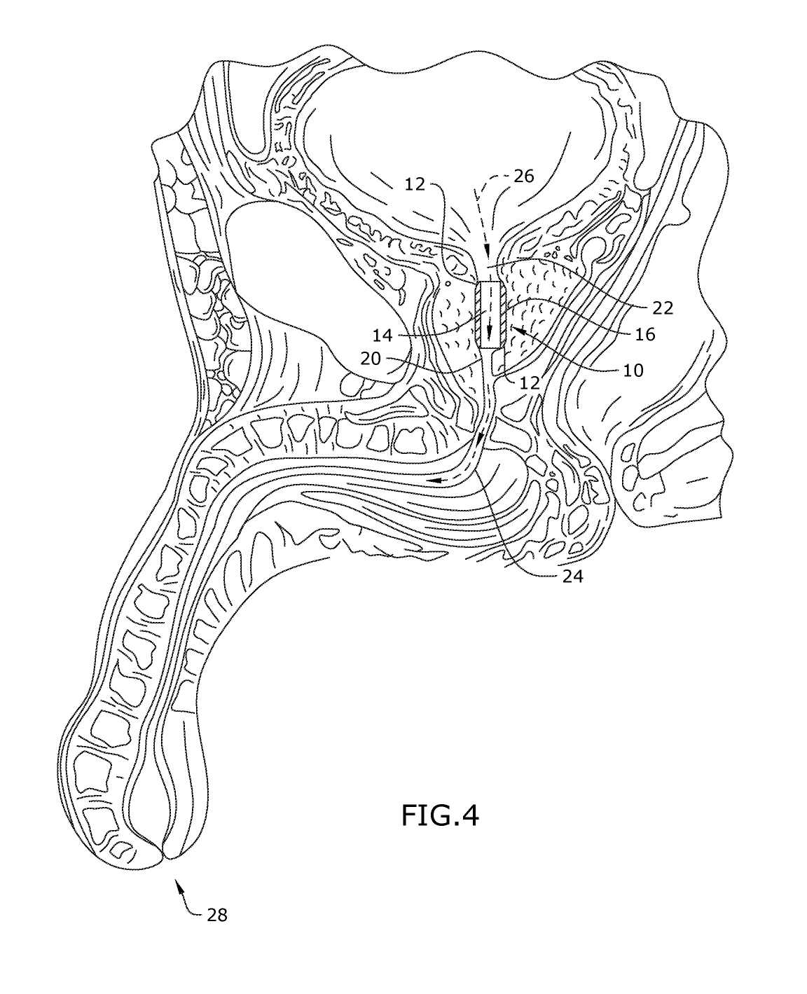

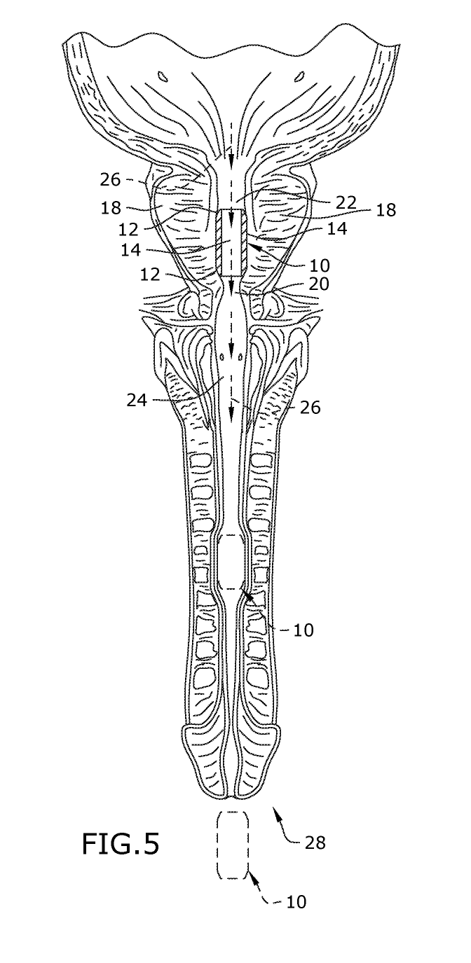

Referring to FIGS. 4 and 5, the prostatic urethral stent 10 may be dimensioned and adapted for transurethral insertion into the lumen of the internal urethra sphincter 22, through the urethra 24, through the external urethra sphincter 20, and through the external opening of the Glans penis of a user. The prostatic urethral stent 10 may be dimensioned and adapted to be retained within the internal urethra sphincter 22 and/or a portion of the prostatic urethra by the constrictive forces exerted by an enlarged prostate 18. Accordingly, the cylindrical opening 14 may have an approximately 7 mm diameter; the uniform circumference of the tubular body 16 may be approximately 9 mm in diameter; and the overall length, from first end 11 to second end 13, may be approximately between 3.0 to 3.25 cm. Given the foregoing dimensions, the prostatic urethral stent 10 may be made of material that is sufficient rigid and strong to resist the constrictive forces exerted by the enlarged prostate 18, while also being uric acid resistant—such material may be titanium or the like.

In certain embodiments, because of its non-elongated shape, the prostatic urethral stent 10 need not be sufficiently flexible to accommodate the natural anatomical bend.

A method of using the present invention may include the following. The prostatic urethral stent 10 disclosed above may be provided. A medical practitioner may surgically and/or endoscopically provide the transurethral insertion of the prostatic urethral stent 10 within the internal urethra sphincter 22 and/or a portion of the prostatic urethra to reinforce the lumen thereof.

It should be understood, of course, that the foregoing relates to exemplary embodiments of the invention and that modifications may be made without departing from the spirit and scope of the invention as set forth in the following claims.

what is claimed is:

1. A prostatic urethral stent comprising:
a tubular body extending between opposing ends;
a tapered portion near each end, wherein a circumference of each tapered portion decreases as the tapered portion extends toward an associated end; and
a cylindrical opening provided through the tubular body,
wherein the tubular body is formed from a sufficiently rigid, uric acid resistant, and non flexible material.

2. The prostatic urethral stent of claim 1 dimensioned and adapted for transurethral insertion into a lumen of an internal urethra sphincter of a human user.

3. The prostatic urethral stent of claim 2 dimensioned and adapted for retention within the lumen of the internal urethra sphincter of the human user, in part, based on a constrictive force of an enlarged prostate of the human user.

4. The prostatic urethral stent of claim 3, wherein a diameter of the cylindrical opening is approximately 7 millimeters.

5. The prostatic urethral stent of claim 4, wherein a circumferential diameter of the tubular body is approximately 9 millimeters.

6. The prostatic urethral stent of claim 5, wherein a length of the tubular body ranges between approximately 3.00 to 3.25 centimeters.

7. The prostatic urethral stent of claim 6, wherein the sufficiently rigid, uric acid resistant, and non flexible material is titanium.

8. A prostatic urethral stent comprising:
a tubular body extending between opposing ends for a length that ranges between approximately 3.00 to 3.25 centimeters, wherein a circumferential diameter of the tubular body is approximately 9 millimeters;
a tapered portion near each end, wherein a circumference of each tapered portion decreases as the tapered portion extends toward an associated end so that each end has a cone shape; and
a cylindrical opening provided through the tubular body, wherein the cylindrical opening has a diameter of approximately 7 millimeters,
wherein the tubular body is formed from titanium, and wherein the tubular body is dimensioned and adapted for transurethral insertion into and retention within a lumen of an internal urethra sphincter of a human user.

9. A method of non-surgically regulating a urethral flow through a prostatic urethra, comprising the step of:
transurethrally inserting a prostatic urethral stent of claim 8 into a lumen of an internal urethra sphincter of a human user.
